(19) United States
(12) Patent Application Publication
(10) Pub. No.: US 2023/0324323 A1
(43) Pub. Date: Oct. 12, 2023

(54) SYSTEMS AND METHODS FOR ALCOHOL CONCENTRATION MONITORING

(71) Applicant: Roth River, Inc., Prospect, KY (US)

(72) Inventors: Jeffrey Thomas Cesnik, Winchester, VA (US); David Robert Durand, Prospect, KY (US); Todd Bryan Pritts, Prospect, KY (US)

(21) Appl. No.: 18/131,353

(22) Filed: Apr. 5, 2023

Related U.S. Application Data
(60) Provisional application No. 63/327,833, filed on Apr. 6, 2022.

(51) Int. Cl. G01N 27/22; G01N 33/14

(52) U.S. Cl. CPC G01N 27/226; G01N 33/146

(57) ABSTRACT

Systems and methods for determining an alcohol by volume (ABV) measurement of liquids that are stored in containers in a storage facility are provided. The ABV is determined by measuring capacitance using one or more electrodes coupled to an outside wall of the storage container. Other environmental parameters can be taken into account, such as a temperature and humidity.

### CROSS-REFERENCE TO RELATED APPLICATIONS

This application claims the benefit of U.S. Ser. No. 63/327,833, filed on Apr. 6 2022, entitled SYSTEMS AND METHODS FOR ALCOHOL CONCENTRATION MONITORING, the disclosure of which is incorporated herein by reference.

### BACKGROUND

Various types of liquids may be stored in containers, whether during production, processing, transportation, distribution, sale, or consumption. For example, during the production of wine, beer, or other types of alcohol and/or spirits, the liquid may be stored in a barrel for an extended period of time, which may range from several months to a number of years. During storage in the barrel, the liquid may undergo a process of fermentation, or aging, in preparation for eventual sale, distribution, and/or consumption.

The barrel, or other type of container, may be made of wood, of which oak is a common element for a variety of alcohol types, or other materials. Certain types of containers may not be completely air tight (whether by design, or by limitation) and a certain amount of liquid may escape, evaporate, leak, or otherwise decrease by volume over time. For example, a wood barrel may absorb a certain amount of the liquid over time, may be constructed of a porous wood that allows for the liquid to evaporate over time, or may include small cracks or openings that allow the liquid to leak out of the container.

A storage facility, such as a rackhouse, rickhouse, dunnage house, or any other type of warehouse, may store a high volume of barrels. Each barrel has a particular volume of liquid that is exposed to various environmental conditions. By way of example, barrels stored on a higher tier of a storage facility may experience more temperature fluctuations than barrels stored at a lower tier. Additionally, other environmental conditions can affect the barrels and the rate of liquid loss throughout a storage facility, such as humidity or barometric pressure. It would be desirable to monitor the environmental conditions, liquid levels, and other conditional parameters of liquid storage containers in a storage facility.

### BRIEF DESCRIPTION OF THE DRAWINGS

It is believed that certain embodiments will be better understood from the following description taken in conjunction with the accompanying drawings, in which like references indicate similar elements and in which:

FIG. 1 schematically depicts the monitoring of various parameters within a hazardous location in accordance with one non-limiting embodiment based on information received from wireless sensor nodes within the location.

FIG. 2 schematically depicts the monitoring of various parameters within a location in accordance with one non-limiting embodiment.

FIG. 3 depicts an example logical and physical architecture of a cloud-based service and a physical storage location in accordance with one non-limiting embodiment.

FIG. 4 depicts an example block diagram of a portion of a monitoring system in accordance with one non-limiting embodiment.

FIG. 5 schematically depicts the identification of a monitored container in accordance with one non-limiting embodiment.

FIG. 6 is an isometric view of an example sensor system in accordance with one non-limiting embodiment.

FIG. 7 is a side view of the example sensor system of FIG. 6.

FIG. 8 is an exploded view of the example sensor system of FIG. 6.

FIG. 9 depicts the installation of an example sensor system onto a wooden storage container in accordance with one non-limiting embodiment.

FIGS. 10A-10B are isometric views of an example sensor system having attachment tabs in accordance with one non-limiting embodiment.

FIG. 11 is an isometric view of an example sensor system having adhesive attachment strips in accordance with one non-limiting embodiment.

FIG. 12 is an isometric view of an example receiver in accordance with one non-limiting embodiment.

FIG. 13 is an exploded view of the example receiver of FIG. 12.

FIG. 14 is an example block diagram of the receiver of FIG. 12.

FIG. 15 schematically depicts the determination of an alcohol concentration of a stored liquid in accordance with one non-limiting embodiment.

FIG. 16 graphically illustrates a capacitance/environmental conditions model in accordance with one non-limiting embodiment.

FIG. 17 schematically depicts the determination of the alcohol concentration of a stored liquid at a first time and a second in accordance with one non-limiting embodiment.

### DETAILED DESCRIPTION

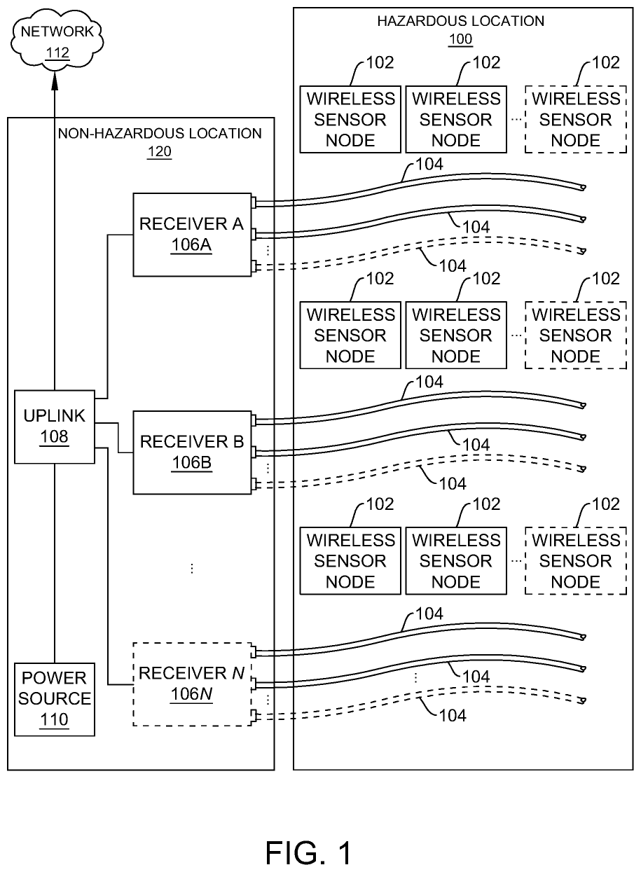

Various non-limiting embodiments of the present disclosure will now be described to provide an overall understanding of the principles of the structure, function, and use of systems, apparatuses, devices, and methods disclosed. One or more examples of these non-limiting embodiments are illustrated in the selected examples disclosed and described in detail with reference made to FIGS. 1-17 in the accompanying drawings. Those of ordinary skill in the art will understand that systems, apparatuses, devices, and methods specifically described herein and illustrated in the accompanying drawings are non-limiting embodiments. The features illustrated or described in connection with one non-limiting embodiment may be combined with the features of other non-limiting embodiments. Such modifications and variations are intended to be included within the scope of the present disclosure.

The systems, apparatuses, devices, and methods disclosed herein are described in detail by way of examples and with reference to the figures. The examples discussed herein are examples only and are provided to assist in the explanation of the apparatuses, devices, systems and methods described herein. None of the features or components shown in the drawings or discussed below should be taken as mandatory for any specific implementation of any of these apparatuses, devices, systems or methods unless specifically designated as mandatory. For ease of reading and clarity, certain components, modules, or methods may be described solely in connection with a specific figure. In this disclosure, any identification of specific techniques, arrangements, etc. are either related to a specific example presented or are merely a general description of such a technique, arrangement, etc. Identifications of specific details or examples are not intended to be, and should not be, construed as mandatory or limiting unless specifically designated as such. Any failure to specifically describe a combination or sub-combination of components should not be understood as an indication that any combination or sub-combination is not possible. It will be appreciated that modifications to disclosed and described examples, arrangements, configurations, components, elements, apparatuses, devices, systems, methods, etc. can be made and may be desired for a specific application. Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment, or “in an embodiment” in places throughout the specification are not necessarily all referring to the same embodiment. Furthermore, the particular features, structures or characteristics may be combined in any suitable manner in one or more embodiments.

Throughout this disclosure, references to components or modules generally refer to items that logically can be grouped together to perform a function or group of related functions. Like reference numerals are generally intended to refer to the same or similar components. Components and modules can be implemented in software, hardware, or a combination of software and hardware. The term “software” is used expansively to include not only executable code, for example machine-executable or machine-interpretable instructions, but also data structures, data stores and computing instructions stored in any suitable electronic format, including firmware, and embedded software. The terms “information” and “data” are used expansively and includes a wide variety of electronic information, including executable code; content such as text, video data, and audio data, among others; and various codes or flags. The terms “information,” “data,” and “content” are sometimes used interchangeably when permitted by context. It should be noted that although for clarity and to aid in understanding some examples discussed herein might describe specific features or functions as part of a specific component or module, or as occurring at a specific layer of a computing device (for example, a hardware layer, operating system layer, or application layer), those features or functions may be implemented as part of a different component or module or operated at a different layer of a communication protocol stack. Those of ordinary skill in the art will recognize that the systems, apparatuses, devices, and methods described herein can be applied to, or easily modified for use with, other types of equipment, can use other arrangements of computing systems, and can use other protocols, or operate at other layers in communication protocol stacks, then are described.

As described in more detail below, the present disclosure generally relates to detection, monitoring, and reporting of various environmental and conditional parameters. While many of the following examples are described in the context of bourbon production for the purposes of illustration, this disclosure is not so limited. Instead, the systems, apparatuses, devices, and methods described herein can be applicable to a variety of instances in which liquid is stored in a container, such as during whiskey production and wine production, for example. Moreover, beyond consumable liquids, the systems, apparatuses, devices, and methods described herein are also applicable to the level detection, monitoring, and reporting of any liquid that is stored in a container, such as chemicals, oils, or industrial liquids. Thus, while many of the examples described herein relate to bourbon barrels, it is to be readily appreciated that the systems, apparatuses, devices, and methods can have applicability across a variety of different types of storage tanks, vessels, and the like. Moreover the systems, apparatuses, devices, and methods described herein can be deployed in a variety of different types of storage facilities, such as warehouses, rickhouses, rackhouses, palletized warehouses, and so forth.

FIG. 1 schematically depicts the monitoring of various parameters within a hazardous location 100 based on information received from wireless sensor nodes 102 within the location. In some embodiments, the hazardous location 100 is an area in which ignitable flammable gases or vapors exists (i.e., a Class 1 Division 1 explosive atmosphere under the 2020 National Electrical Code (NEC or NFPA 70)). Class I locations consist of areas where gases, vapors or liquids may exist that have the potential to become flammable or ignitable. There are two different divisions that exist in Class I, Division 1 and Division 2, along with three Zones; Zone 0, 1 & 2. Locations that would be considered to be Class I include Petroleum Refineries, Gasoline Storage Areas, Dry Cleaning Plants, Spray Finishing Areas, Fuel Servicing Areas, etc. Division 1 is a subset of Class I and is classified as an area where the explosive or flammable gases, vapors or liquids mentioned above can exist under normal, everyday operating conditions.

Upon consideration of this disclosure, however, it is to be appreciated that the systems and methods described herein can be used in a variety of operational environments beyond hazardous locations.

As described in more detail below, a plurality of wireless sensor nodes 102 can be positioned within the hazardous location 100 that are configured to periodically broadcast sensor-based information. In some embodiments, each wireless sensor node 102 is associated with a barrel of stored liquid (such as distilled spirits, for example) and can be configured to monitor several parameters that occur inside and outside a barrel. These measurements can include liquid level, ambient temperature, ambient pressure, humidity, acceleration (barrel movement), among others. The wireless sensor nodes 102 can also wirelesses relay battery information, such as the state of charge and the remaining capacity, during its periodic wireless transmission routine.

As schematically shown in FIG. 1, one or more radiating cables 104 can be placed in the hazardous location 100. The radiating cables 104 can be any suitable type of antenna that collects signals broadcasted from the wireless sensor nodes 102. In some embodiments, the radiating cables 104 can be a coax cable with a central conductor and a perforated shielding (sometimes referred to as “leaky feeders”) that are routed throughout the hazardous location 100 proximate to the wireless sensor nodes 102. The perforations of the shielding allows the signals generated by the wireless sensor nodes 102 to be received by the central conductor. While the length of each radiating cable 104 can vary based on implementation parameters, in some cases the radiating cable 104 can be between about 5 feet long, about 10 feet long, about 20 feet long, about 30 feet long, about 40 feet long, about 50 feet long, or more than 50 feet long, for example. In some embodiments the radiating cable can be between 100 and 500 feet long, for example. In some implementations using a plurality of different radiating cables, each radiating cable can be the same length or a different length. They can be placed in a zigzag pattern, for example, throughout the hazardous location 100 (such as up and down aisles) to collect signals from the various wireless sensor nodes 102 positioned along the aisles. Depending on the arrangement of the hazardous location 100, the radiating cables 104 can also be placed on the ceiling, on the walls, along support structure, and so forth.

Each of the radiating cables 104 can be in electrical communication with one of the receivers 106A-N. In some embodiments, as depicted in FIG. 1, the receivers 106A-N can be positioned in a non-hazardous location 120. As is to be appreciated any suitable number of radiating cables 104 can be connected to each receiver 106A-N. In some embodiments, the receivers 106A-N are positioned in a weatherproof enclosure that is suitable for mounting outdoors and operating from −40° C. to 85° C., although this disclosure is not so limited. By way of example, the receivers 106A-N can be mounted external to a rickhouse, or other storage, facility.

Still referring to FIG. 1, an uplink 108 can also be positioned in the non-hazardous location 120. The uplink 108 can serve to transmit the information provided by the wireless sensor nodes 102 to a network 112. In some embodiments, the uplink 108 can include an antenna that allows for dual band, LTE cellular and WiFi transmissions, for example. Further, an enclosure of the uplink 108 can be climate controlled and receive power from a power source 110. The power received from the power source 110 can also be provided to the receivers 106A-N through a power over Ethernet (PoE), for example. Generally, the uplink 108 can send signals collected from a rickhouse, or other type of storage site, up to the Internet for further processing, analytics, and data storage. Moreover, the uplink 108 can provide an uplink connection for all of the receivers 106A-N deployed at a site.

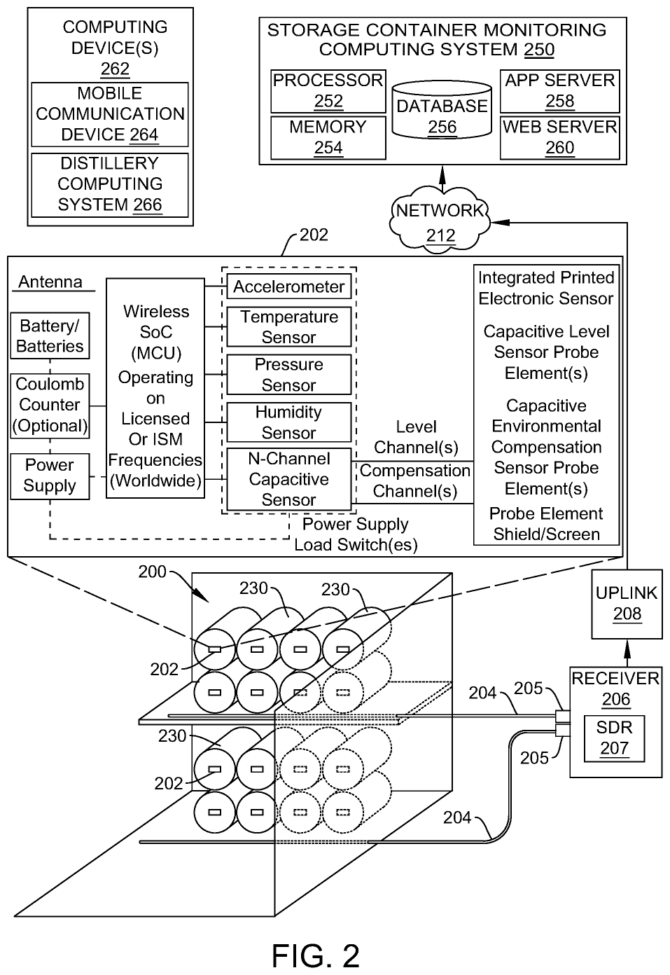

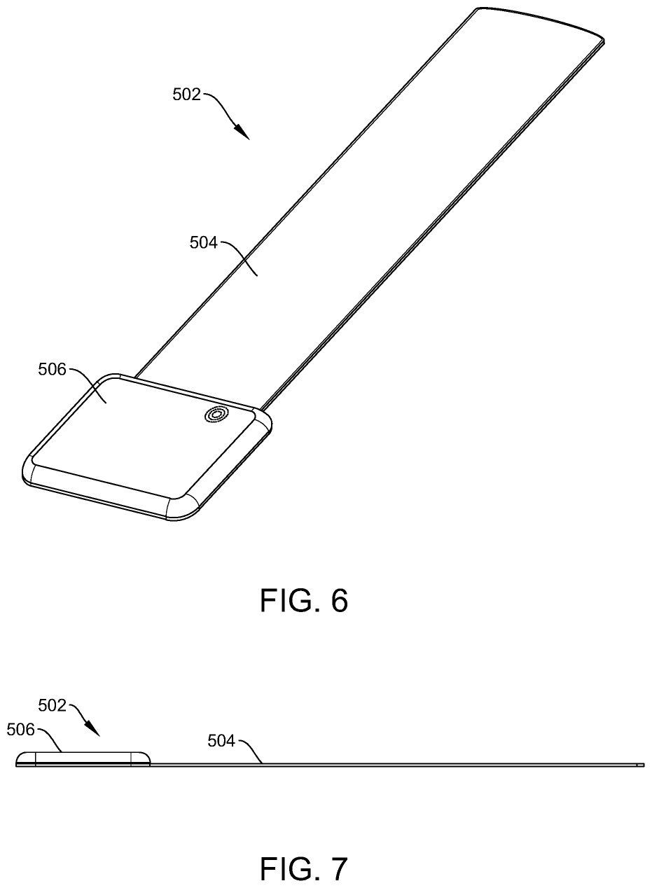

FIG. 2 schematically depicts the monitoring of various parameters within a location 200 in accordance with one non-limiting embodiment. A plurality of storage containers 230 are shown being stored in the location 200. The storage container 230 can be configured to store a liquid over a period of time. As is to be appreciated, the length of the period of time can vary based on the type of liquid, but in some embodiments the length of time is months, years, or even decades. FIG. 2 shows the storage containers 230 in a horizontal position, although other storage containers may be stored in a vertical position (i.e., palletized) without departing from the scope of the present disclosure. Each of the storage containers 230 can be associated with a wireless sensor node 202. A plurality of radiating cables 204 are shown routed through the location 200 such that information broadcasted by the wireless sensors nodes 202 can be collected by one or more radiating cables 204 and provided to a receiver 206. FIG. 2 also schematically depicts an example logical architecture of the wireless sensor node 202. Additional details regarding example wireless sensor nodes 202 can be found below with reference to FIGS. 6-11.

In some embodiments, a microcontroller of each wireless sensor node 202 transmits data through a 2.4 gigahertz ISM band radio signal. However, data can be transmitted on any frequency with a transceiver including 900 megahertz, 400 megahertz, 2.4 gigahertz, and 5 gigahertz, for example. Additionally, with regard to rickhouses, the stored barrels can be viewed as large walls of water between every row. Radio frequencies do not propagate through water well, if at all, and attenuate quite sharply. While a lower frequency radio could potentially transmit through these walls of water, the 2.4 gigahertz signal is generally completely blocked by water. This transmission issue is bypassed through the implementation of a distributed antenna system of the present disclosure as the radio frequency transmissions are collected proximate to each wireless sensor node.

As the radiating cables 204 can be routed through an explosive environment, the receiver 206 can have intrinsically safe radio frequency (RF) isolators 205. The receiver 206 can include a software defined radio (SDR) 207 and a processor. In some example embodiments, the SDR 207 is a two channel wide-band receiver that allows for radio signals from the wireless sensor nodes 202 to be received, decoded, and de-packetized. When the location 200 can have a large number of wireless sensors nodes 202 that are all transmitting on the same frequency, it is possible to encounter issues where the transmissions overlap. To combat this, radio frequencies can be separated. In some embodiments, between 2-80 channels, each channel being 1-2 megahertz wide, can be utilized. While a normal radio receiver can only listen to one channel at a time, the wide-band software defined radio 207 can beneficially listen to multiple channels all at the same time. As a result, one radio transceiver can be used to simultaneously decode transmissions coming from multiple channels inside the location 200.

As shown, the receiver 206 can provide the transmissions to the uplink 208 which can, for example, provide transmissions to a storage container monitoring computing system 250 via a network 212. The storage container monitoring the computing system 250 can provide information to various computing devices 262, such as a mobile communication device 264 or a distillery computing system 266. Such information can relate to, for example, volume levels of various containers 230, environmental data associated with various containers 230, and environmental data associated with the location 200. In some embodiments, information regarding the structural soundness of the location 200 can be ascertained by the data received from the wireless sensor nodes 202. For example, slight movements of the wireless sensor nodes 202 over time can be tracked to identify if the barrels are starting to lean or otherwise shift, which can be indicative of a pending collapse or other structural failure of the rickhouse.

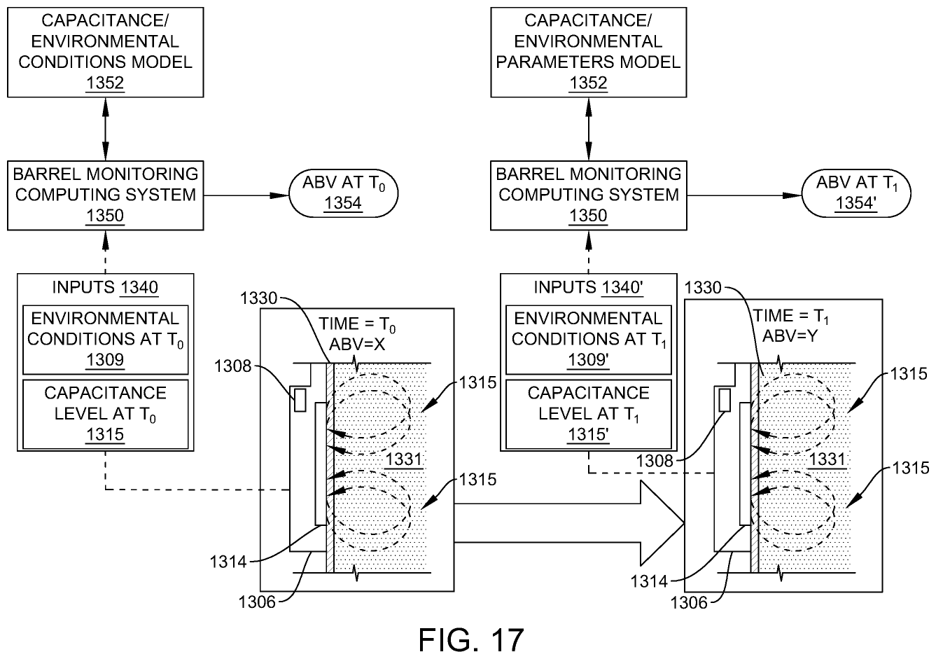

The storage container monitoring computing system 250 can include one or more processors 252 configured to execute code stored in memory 252. Data collected from various barrels can be stored in various types of data stores, schematically shown as database 256. The storage container monitoring computing system 250 can further include one or more computer servers, which can include one or more web servers, one or more application servers, and/or other types of servers. For convenience, only one web server 260 and one application server 258 are depicted in FIG. 17, although one having ordinary skill in the art would appreciate that the disclosure is not so limited. The servers 258, 260 can cause content to be sent to the computing devices 262, or other computing devices, via a network in any of a number of formats. The servers 258, 260 can be comprised of processors (e.g. CPUs), memory units (e.g. RAM, ROM), non-volatile storage systems (e.g. hard disk drive systems), and other elements. The servers 258, 260 may utilize one or more operating systems including, but not limited to, Solaris, Linux, Windows Server, or other server operating systems.

In some embodiments, the web server 258 can provide a graphical web user interface through which various users can interact with the storage container monitoring computing system 250. The graphical web user interface can also be referred to as a graphical user interface, client portal, client interface, graphical client interface, and so forth. The web server 260 can accept requests, such as HTTP requests, from clients and serve the clients responses, such as HTTP responses, along with optional data content, such as web pages (e.g. HTML, documents) and linked objects (such as images, video, documents, data, and so forth). The application server 258 can provide a user interface for users who do not communicate with the storage container monitoring computing system 250 using a web browser. Such users can have special software installed on their computing device to allow the user to communicate with the application server 258 via a network.

The storage container monitoring computing system 250 can be in communication with the containers 230 via the network 212, using a suitable communications interface. The network 212 can be an electronic communications network and can include, but is not limited to, the Internet, LANs, WANs, GPRS networks, other networks, or combinations thereof. The network 212 can include wired, wireless, fiber optic, other connections, or combinations thereof. In general, the network 212 can be any combination of connections and protocols that will support communications between the storage container monitoring computing system 250 and the wireless sensors nodes 202. In some embodiments, the wireless sensor nodes 202 provide raw data and the storage container monitoring computing system 250 performs analysis on the data to assess volume change, environmental conditions, and so forth.

Embodiments of the storage container monitoring computing system 250 can also be implemented in cloud computing environments. “Cloud computing” may be defined as a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

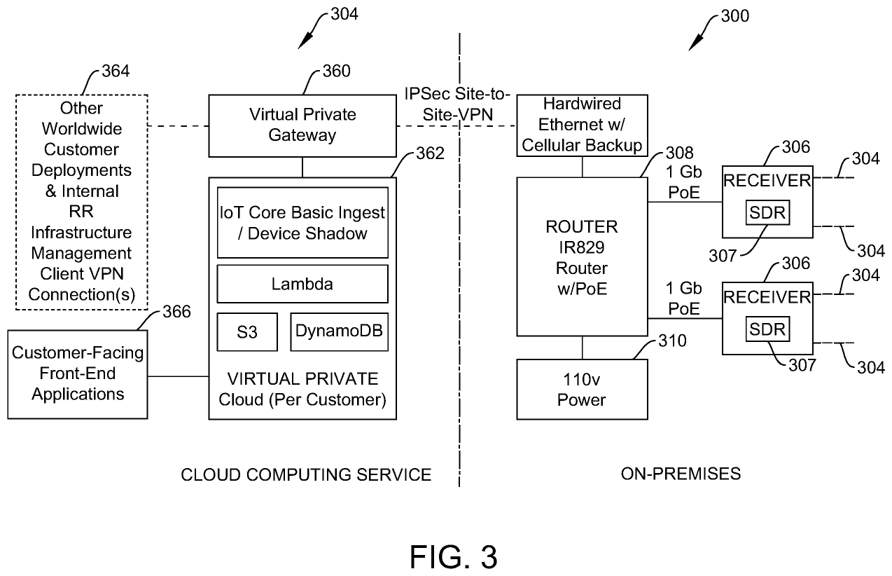

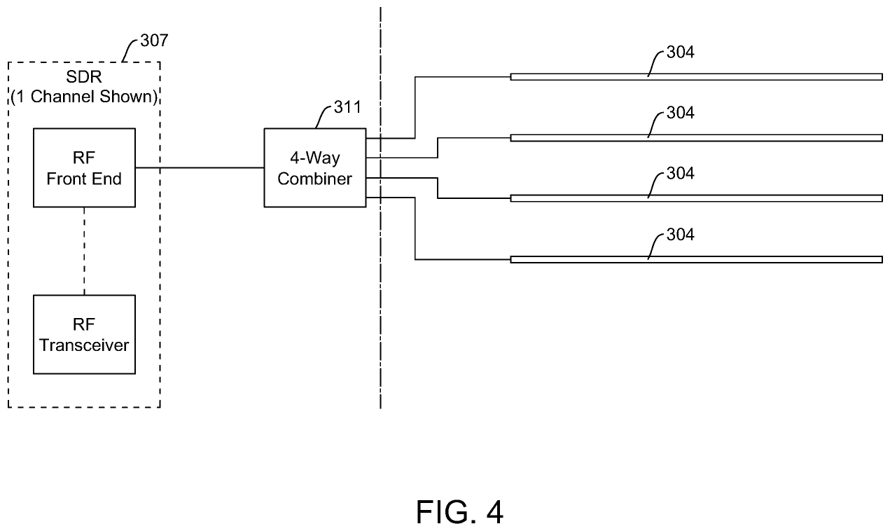

FIG. 3 depicts an example logical and physical architecture of a cloud-based service 304 and a physical storage location 300 in accordance with one non-limiting embodiment. As shown, radiating cables 204 at the physical storage location 300 are in electrical communication with a receiver 306 having a SDR 307. In this embodiment, the receivers 306 are in communication with an uplink 308 via an Ethernet PoE (Power over Ethernet) connection. The uplink 308 can be powered by a 110V power source 310. The uplink 308 can communicate via a virtual private network (VPN), for example, to a virtual private cloud 302. Customer-facing front-end applications 366 can present data, provide visualizations, dashboards, metrics, and the like. Additionally, FIG. 3 depicts the collection of data from other physical storage locations at other deployments 364. FIG. 4 depicts an example block diagram of a portion of the example logical and physical architecture of a cloud-based service 304 in accordance with another one non-limiting embodiment. In this example embodiment, a 4-way combiner 311 is positioned between the radiating cables 304 and the SDR 307. As is to be appreciated, however, a wide variety of logical and physical architectures can be deployed without departing from the scope of the present disclosure.

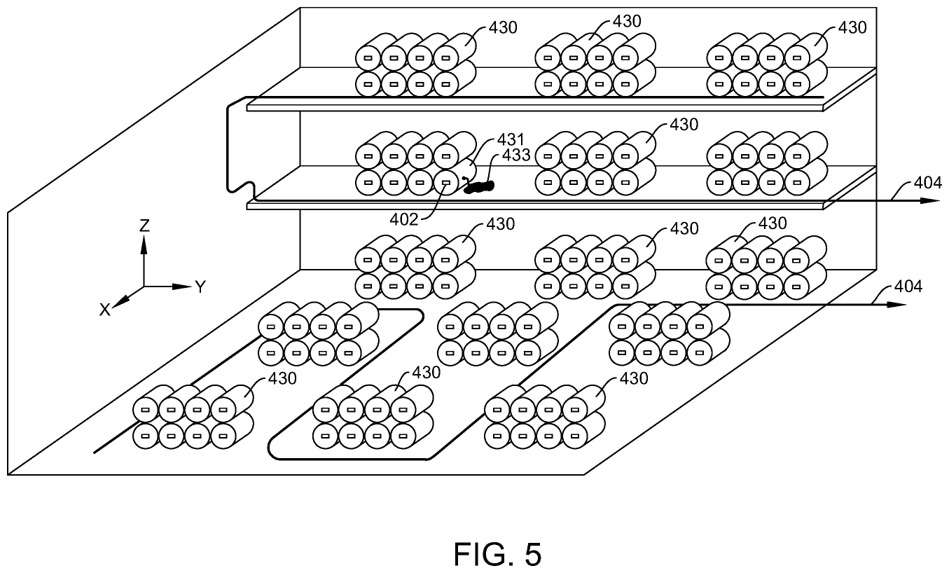

FIG. 5 schematically depicts the identification of a particular monitored container in accordance with one non-limiting embodiment. As shown, a plurality of storage containers 430 are shown that are each associated with a respective wireless sensor node 402. In some embodiments, each wireless sensor node 402 collects pressure data that can be used as an altimeter to detect the height (i.e., in the Z axis) of each storage container 430. By way of example, all storage containers 430 that are at the lowest level in the storage facility should detect a higher pressure data than the storage containers 430 that are positioned on shelfs, or on higher floors. Additionally, in some embodiments static sensors could also be deployed to set baseline pressure readings for the floor and ceiling of the storage facility. Based on the upper and lower pressure readings within a storage location, the height of any wireless sensor node 402 can be extrapolated based on its pressure reading.

With regard to determining the location of a particular storage container 430 in the X-Y plane, any of a variety of techniques can be used. In one embodiment, the radio receiver that is picking up the strongest signal from the particular storage container 430 can be determined. As the signal collected from wireless sensor node 404 collected by the radiating cable 404 naturally loses signal strength as it travels toward the receiver, and based on the known routing of the radiating cable through the storage location, it can be determined where the storage container is along the radiating cable by its signal strength. Therefore, through a combination of pressure readings and signal strength determination, the location of a particular storage container to be geolocated in the X-Y-Z dimensions.

By way of an operational example, the storage container 431 in FIG. 5 is shown to be leaking product 433. The wireless sensor node 402 monitoring that storage container 431 can broadcast a signal that is collected by the radiating cable 404 that is indicative of liquid level. The location of the leaking storage container 431 can then be ascertained based on the ambient pressure reading collected by the wireless sensor node 402 as well as the strength of the signal from the wireless sensor node 402 collected at the receiver, in addition to the known distinct radiating cable run attached to the receiver (not shown). Accordingly, it can be determined, for example, that the leaking storage container 431 is 20 feet above the floor of the storage facility. The strength of the signal can be used to determine that the wireless sensor node 402 of the leaking storage container 431 was transmitted along the radiating cable 404 about 100 feet. Based on that information, a technician can locate the leaking storage container 431 and address the issue. An additional embodiment that uses a humidity sensor can also optionally be used to identify leaks proximate to a given sensor. That is, if a sensor or group of sensors detects a localized humidity increase relative to the rest of the rickhouse it could be determined that a leak is close by. As is to be appreciated, a wide array of event detection and monitoring can be implemented based on the systems and methods disclosed herein.

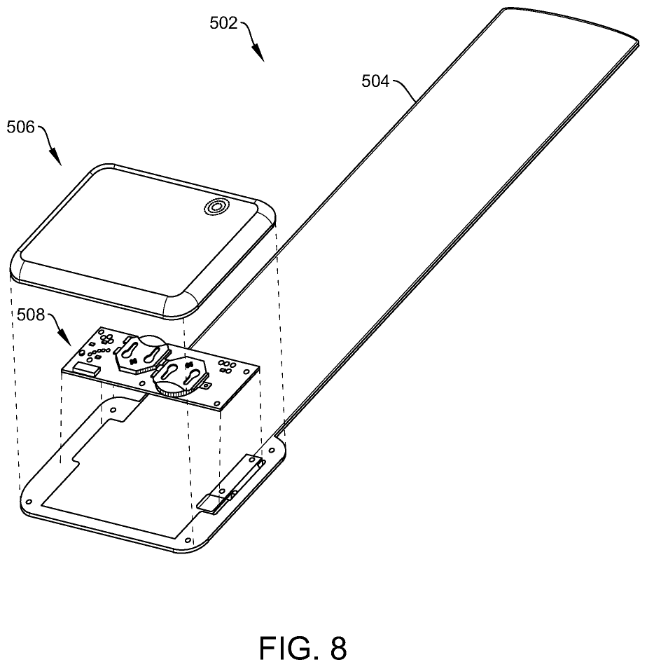

FIG. 6 is an isometric view of an example sensor system 502 in accordance with one non-limiting embodiment, FIG. 7 is a side view of the example sensor system of FIG. 6, and FIG. 8 is an exploded view of the example sensor system of FIG. 6. Generally, the sensor system 502 can function as a wireless sensor node 102, 202, 402, as described above, and collect and transmit measurements such as liquid level, ambient temperature, ambient pressure, humidity, and acceleration (barrel movement). Sensors of the sensor system 502 can also relay battery information such as the state of charge and the remaining capacity. While the structure of the sensor system 502 can vary, in some embodiments, the sensor system 502 comprises an electrode portion 504 that is coupled to a sensor housing 506. The electrode portion 504 can contain an electrode array that can be used to determine a volume level and the sensor housing 506 can include various sensors, as described herein. Furthermore, in accordance with some embodiments, the sensor system 502 can last 8-12 years or more and have an extremely low power demand.

A first side of the electrode portion 504 can contain a plurality of electrodes with ground shielding positioned therebetween. A total ground shield can be positioned on the second side. In one embodiment, the electrode portion 504 includes PET (polyethylene terephthalate) semicrystalline, which is a type of alcohol resistant plastic. Other plastic or non-conductive formulations (e.g. polycarbonate, fiberglass, etc.) could also be used in the construction of alternative embodiments. The electrode portion 504 can include two sensing electrodes and a static reference electrode. The two sensing electrodes are engineered to project an electric field through the wood of a barrel. The static electrode compensates the other two electrodes for ambient moisture and temperature effects. The main level sensing electrodes of the sensor system 502 can measure liquid levels within a barrel and account for capacitance change while ignoring ambient effects as measured by the static electrode. The sensor housing 506 can include an electronics package 508 (FIG. 8) that can include a capacitive sensing chip connected to the sensing elements in the electrode portion 504. The electronics package 508 can also contain a microcontroller which is a collection device that can “wake up” on a pre-programmed schedule to take a series of measurements and wirelessly transmit the collected data (i.e., to a radiating cable positioned proximate to the sensor system 502). The electronics package 508 can be powered by one or two coin-cell batteries, or other suitable power source. The electrode portion 504 can be manufactured using a variety of suitable techniques. For example, in one embodiment, printed electronics are laminated to a core piece of plastic. In other embodiments, electronics can be printed directly on top of a plastic substrate and a circuit board can be attached to the top of the sensor with conductive adhesive, anisotropic conductive film (ACF), or a low-temperature soldering process, for example, which allows the board to be stuck down with or without adhesive while still allowing for electrical contact with the silver-printed electrodes. Further, other embodiments could implement electrodes directly on the printed circuit board itself.

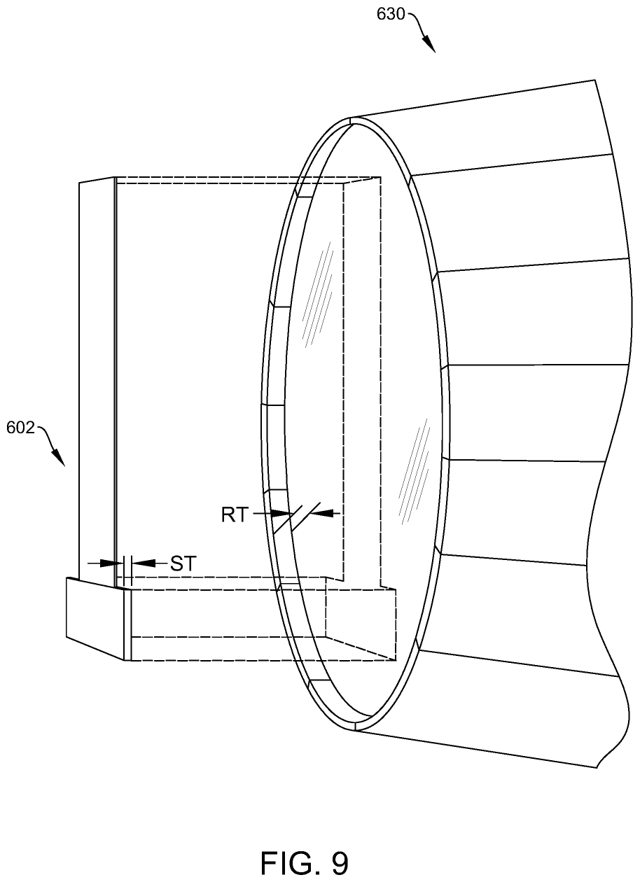

FIG. 9 depicts the installation of the example sensor system 602 onto an example wooden storage container 630 in accordance with one non-limiting embodiment. In some embodiments, the sensor system 602 is low profile such that its maximum thickness (shown as ST) is less than or equal to the thickness of rim of the wooden storage container 630 (shown as RT). In one example embodiment the ST is less than ⅜ inch. When the storage container 630 is placed on a rack for storage, it may not orientated such that the sensor system 602 is perfectly vertical. Beneficially, the electrode array can compensate for minor misalignments allowing for the sensor system 602 to be vertically offset by a few degrees.

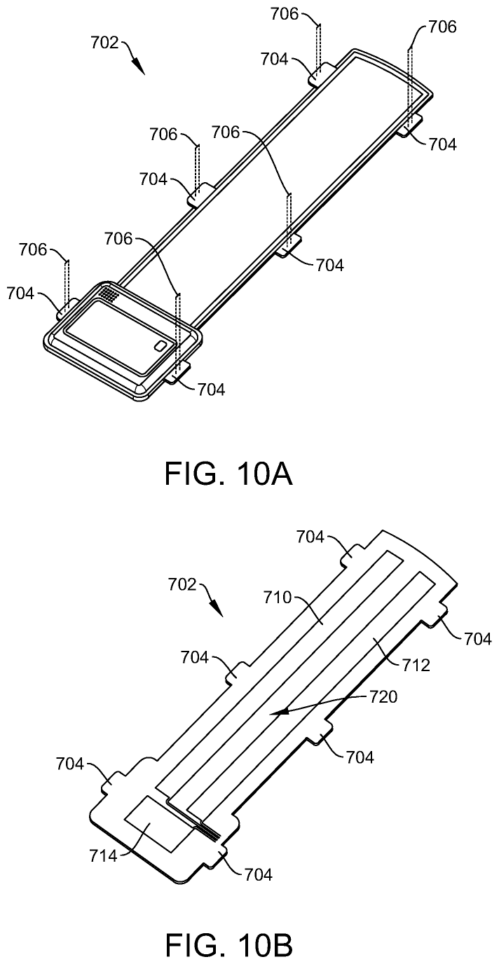

Sensor systems in accordance with the present disclosure can be coupled to a barrel (or other storage container) using any suitable method. For example, FIGS. 10A-10B are isometric views of an example sensor system 702 having attachment tabs 704 in accordance with one non-limiting embodiment. FIG. 10A is a top view of the sensor system 702 and FIG. 18B is a bottom view showing a contacting surface 720. The attachment tabs 704 can extend laterally from either side of the sensor system 702 and receive fasteners 706 (FIG. 10A), such as stainless steel staples, brads, tacks, or the like, to attach the sensor system 702 to a wooden barrel. FIG. 10B depicts an example electrode arrangement comprising a first level sensing electrode 710 and a second level sensing electrode 712. A static electrode 714 can also be utilized to compensate the first and second level sensing electrodes 710 and 712 for ambient moisture and temperature effects, for example. While FIG. 10B illustrates an example electrode configuration, this disclosure is not so limited. As is to be appreciated, a variety of electrode arrangements can be used to focus an electric field into an associated barrel, including any suitable number of electrodes, size of electrodes, layout of electrodes, and type of electrodes. While not shown in FIG. 10B, shielding can also be deployed proximate to the electrodes to aid in focusing the generated electric fields. In one embodiment, the shield could be static (i.e. connected to local battery ground). In other embodiments, the shield could be actively driven (i.e. a driven shield) to potentially increase the sensitivity of the sensor electrodes.

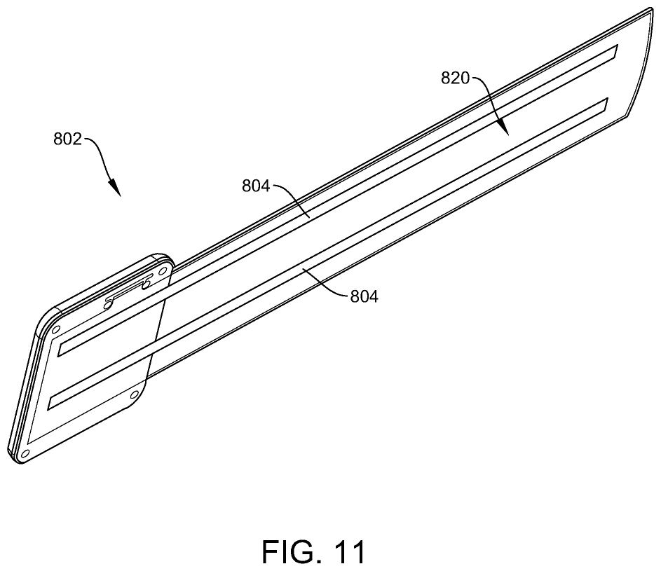

In accordance with another embodiment, FIG. 11 is an isometric view of an example sensor system 802 having adhesive attachment strips 804 positioned on its underside on a contacting surface 820 (the electrodes of sensor system 802 are not shown in FIG. 11). The adhesive attachment strips 804 can be used to adhere the sensor system 802 to a barrel or other storage container As is to be appreciated, however, any suitable technique can be used to permanently or semi-permanently couple a sensor system to a storage container, such as adhesives, brackets, screws, nails, straps, hook and loop fasteners, friction fit, magnets, and the like.

While the sensor system of FIG. 6-11 depict sensor systems that are to be coupled to the flat end of a barrel which are stored horizontally, this disclosure is not so limited. For example, in some embodiments, a sensor system configured for storage containers that are stored in a vertical orientation can be utilized. For example, with regard to barrels that are stored in a vertical orientation (i.e. in a palletized warehouse), a sensor system can be placed on the outside surface of an upright barrel to allow for liquid measurements along the vertical axis. These sensor systems for palletized warehouses can be molded to conform to any curvature of the storage container and to bump over exterior barrel hoops, as may be required. In other embodiments, the sensor system electrodes could be embedded within the construction of the barrel itself.

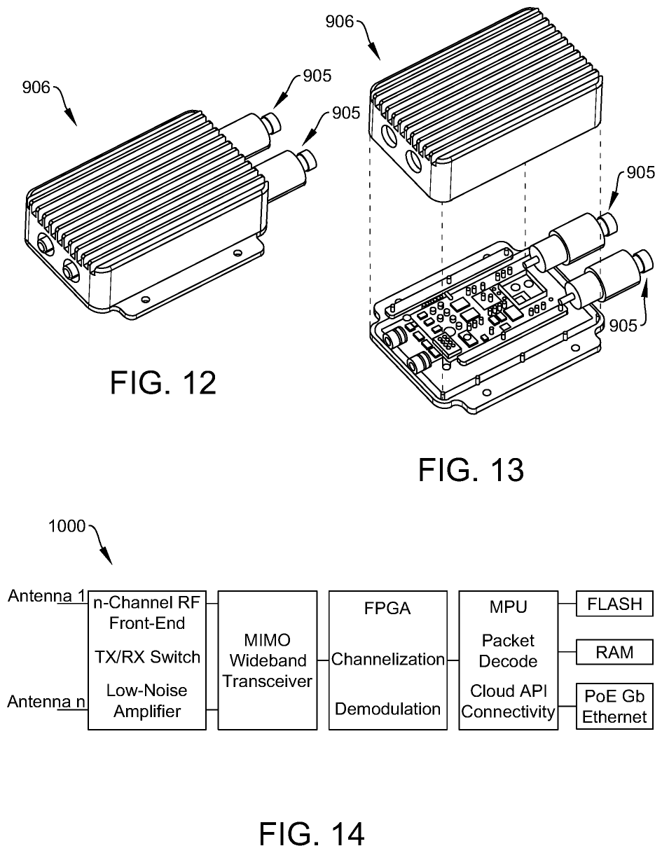

FIG. 12 is an isometric view of an example receiver 906 in accordance with one non-limiting embodiment. FIG. 13 is an exploded view of the example receiver 906 and FIG. 14 is an example block diagram of the logical architecture of the receiver 906. Similar to receivers 106, 206, and 306 described above, the receiver 906 can receive radio frequency signals via radiating cables (not shown) that are connected to intrinsically safe RF isolators 905.

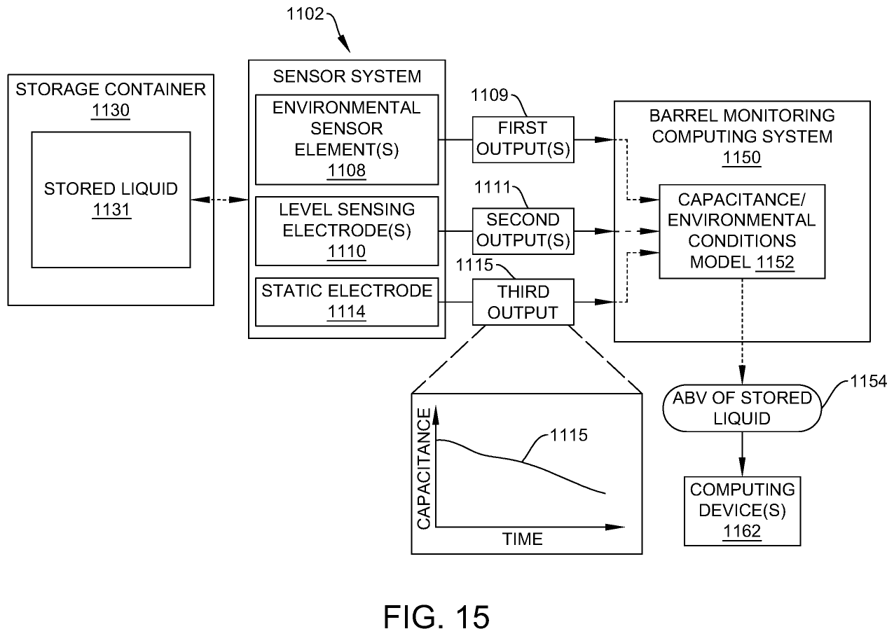

FIG. 15 schematically depicts the determination of an alcohol concentration of a stored liquid 1131 using data collected by a sensor system 1102 in accordance with various embodiments. A storage container 1130 can store the liquid 1131 for an extended period of time and, similar to various embodiments described above, a sensor system 1102 can be coupled to the storage container 1130. The sensor system 1102 can be, for example, a wireless sensor node that includes one or more environmental sensor elements 1108 for measuring various conditions or parameters, such as ambient temperature, ambient pressure, humidity, acceleration, and so forth. The output(s) 1109 of such sensors 1108 can be wirelessly provided to a barrel monitoring computing system 1150 over any suitable network connections, as described above.

The sensor system 1102 can also include various sensor elements usable for determining a liquid level within the storage container 1130, such as level sensing electrode(s) 1110 and a static electrode 1114. The level sensing electrode(s) 1110 can be configured similarly as the first level sensing electrode 710 (FIG. 10B) and a second level sensing electrode 712 (FIG. 10B) and the static electrode 1114 can be configured similarly as the static electrode 714 (FIG. 10B), although this disclosure is not so limited. The outputs 1111 of level sensing electrode(s) 1110 and the output 1115 of the static electrode 1114 can be utilized by the barrel monitoring computing system 1150 to measure a level of the stored liquid 1131, as described above. More specifically, a ratio of the capacitance value measured by the level sensing electrode(s) 1110 and the static electrode 1114 can be used to derive a relative level measurement of the stored liquid 1131 over the various environmental conditions experienced during storage.

In accordance with the present disclosure, one or more outputs from the sensor system 1102 can additionally or alternatively be used to deduce a real-time alcohol concentration of the stored liquid 1131. More specifically, the one or more outputs of the sensor system 1102 can include a capacitance measurement 1115 that changes over time as the alcohol concentration of the stored liquid 1131 changes over time. In accordance with various embodiments, as the water content of the stored liquid 1131 slowly evaporates from the stored liquid 1131 over time, the capacitance readings as collected by the static electrode 1114 and/or the level sensing electrode(s) 1110 of the sensor system 1102 will slowly decrease. The various measurements generated by sensor system 1102 over time, such as capacitance measurements and environmental conditions measurements, can be applied to a capacitance/environmental conditions model 1152 by the barrel monitoring computing system 1150 to ascertain the alcohol concentration 1154 of stored liquid 1131. As schematically illustrated in FIG. 15, the alcohol concentration 1154 as determined based on the capacitance measurement of the stored liquid 1131 and the environmental conditions can be provided to a computing device 1162, such as a mobile communications device or a distillery computing system. Thus, while a ratio of capacitance measurements can be utilized to determine a liquid level (as described above), the absolute measured capacitance value(s) provided by the source electrode(s), in combination with environmental condition data, can be utilized in the automated determination of the alcohol concentration 1154 of the stored liquid 1131.

In some embodiments, the alcohol concentration 1154 of the stored liquid 1131 can be derived from the output 1115 of the static electrode 1114 in combination with the outputs 1109 from one or more of the environmental sensor elements 1108. Alternatively, in other embodiments, the alcohol concentration 1154 of the stored liquid 1131 can be derived from the output from the level sensing electrode(s) 1110 and the static electrode 1114, along with the outputs from one or more of the environmental sensor elements 1108. In yet other embodiments, the alcohol concentration 1154 of the stored liquid 1131 can be derived from the output 1111 of the level sensing electrode(s) 1110 in combination with the outputs 1109 from one or more of the environmental sensor elements 1108.

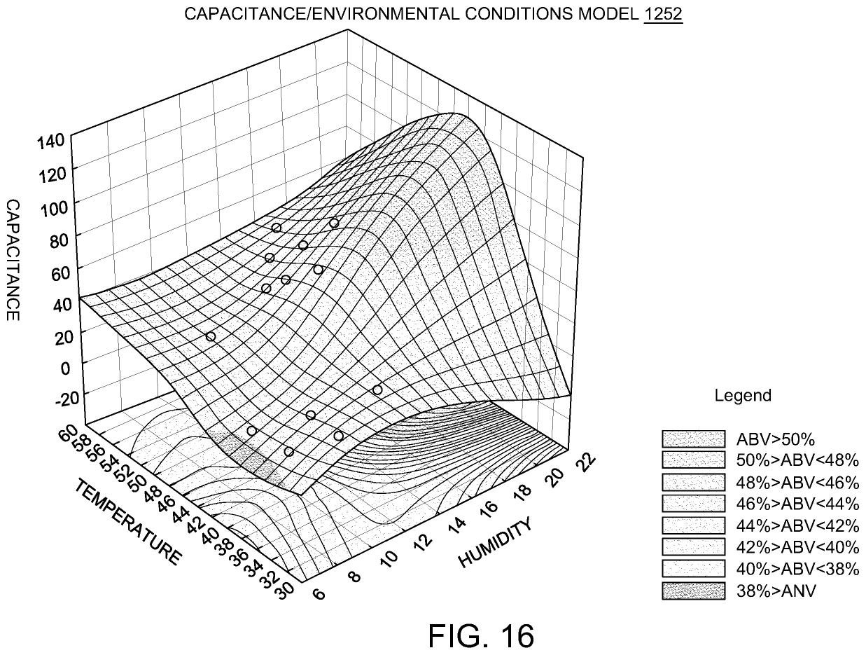

FIG. 16 graphically illustrates a capacitance/environmental conditions model 1252 in accordance with one non-limiting embodiment. This example model is based on, for example, capacitance measurements, temperature measurements, and humidity measurements to determine ABV (alcohol by volume) values of a stored liquid. The capacitance measurement can be collected by one or more electrodes, such as the static electrode 1114 (FIG. 15) and/or the level sensing electrode(s) 1110 (FIG. 15) and the temperature and humidity measurements can be collected by the environmental sensor elements 1108 (FIG. 15). While the model depicted in FIG. 16 utilizes temperature measurements and humidity measurements as inputs, this disclosure is not so limited. Other models in accordance with the present disclosure can utilize less input data (i.e., “capacitance and temperature” or “capacitance and humidity,” for example) or more input data (i.e., “capacitance, temperature, humidity, and pressure,” for example) or different data conditions (i.e., “capacitance, temperature, and pressure” or “capacitance, humidity, and pressure,” for example). Accordingly, this disclosure is not limited to any particular combination of inputs to model, as a variety of suitable models can be used to determine an ABV value based on various sensor readings.

The model 1252 can be developed using any suitable modeling approach, such as empirical measurements or machine learning, using any suitable data set. In some implementations, baseline calibration can be performed using data collected from a large number of storage containers, which are each simultaneously storing the same type of liquid. By way of example, data can be collected from upward of 10,000 storage vessels, 50,000 storage vessels, 1 million storage vessels, or more than 5 million storage vessels for model calibration purposes. Further, a variety of models can be developed that are applicable to different types of stored liquids, different types of storage containers, and so forth. In addition to dynamic input variables, the models can also take into account other constants or semi-constants, such as a dielectric constant of the sidewall of the storage container, the dielectric constant of adhesive used to secure the sensor system to the sidewall, and so forth. In any event, in accordance with the systems and methods described herein, a sample of liquid does not need to be removed from the storage container in order for the barrel monitoring computing system to determine its ABV value, as the ABV value can be determined in situ in in real-time.

Referring now to FIG. 17, the determination of the ABV of a stored liquid 1131 at a first time (shown as T₀) and a second time (shown as Ti) is depicted. The difference between T₀ and T₁ can be any suitable timeframe, such as one day, one week, one month, one year, five years, a decade, or more. An electrode 1314 and a sensor housing 1306 are shown coupled to the outside surface of sidewall 1330 of a storage container that contains the stored liquid 1331. Such configuration can be similar to the arrangement illustrated in FIG. 9, for example. As schematically shown in FIG. 17, the electrode 1314 can project an electric field 1315 through the stored liquid 1131 to measure its capacitance. Additionally, one or more environmental sensors 1308 can also be associated with the sensor housing 1306, which can generate output signals based on various environmental conditions, such as temperature, humidity, pressure, and so forth. Inputs 1340 can be provided to a barrel monitoring computing system 1350 in accordance with the systems and methods described above. As shown, the inputs 1340 can include environmental conditions 1309 (as measured at T₀) and a capacitance level 1315 (as measured at T₀), as measured by one or more environmental sensors 1308 and the electrode 1314. Based on those inputs, the barrel monitoring computing system 1350 can apply a capacitance/environmental conditions model 1352 to determine an ABV 1354 at T₀. Furthermore, while a single electrode 1314 is schematically depicted in FIG. 17 for the purposes of illustration, it is to be appreciated that outputs from a plurality of a different electrodes coupled to the sidewall 1330 can each provide an input to the barrel monitoring computing system 1350 which can be used for the ABV determination. In this regard, the electrode 1314 can be any of a static electrode, level sensing electrode(s), or a combination thereof.

Furthermore, while FIG. 17 schematically depicts the use of a single capacitance/environmental conditions model 1352, this disclosure is not so limited. As provided above, the barrel monitoring computing system 1350 can use any of a variety of different models, which may depend on the type of liquid being stored and the type of storage container. By way of example, a model for a liquid being stored in a charred wooden vessel may be different than the model for a liquid being stored in a non-charred wooden vessel due to the impact of the char on the capacitance reading of the electrode 1314, or combination of electrodes. Similarly, a model for a first liquid being stored in a storage container (i.e., a high rye mash) may be different than the model for a different liquid being stored in the same storage container (i.e., a high wheat mash).

Referring still to FIG. 17, at Ti a new set of inputs 1340′ can be provided to the barrel monitoring computing system 1350. As shown, the inputs 1340′ can include environmental conditions 1309′ (as measured at Ti) and a capacitance level 1315′ (as measured at Ti). Based on those inputs, the barrel monitoring computing system 1350 can apply the capacitance/environmental conditions model 1352 to determine an ABV 1354′ measured at Ti.

The dimensions and values disclosed herein are not to be understood as being strictly limited to the exact numerical values recited. Instead, unless otherwise specified, each such dimension is intended to mean both the recited value and a functionally equivalent range surrounding that value.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The foregoing description of embodiments and examples has been presented for purposes of description. It is not intended to be exhaustive or limiting to the forms described. Numerous modifications are possible in light of the above teachings. Some of those modifications have been discussed and others will be understood by those skilled in the art. The embodiments were chosen and described for illustration of various embodiments. The scope is, of course, not limited to the examples or embodiments set forth herein, but can be employed in any number of applications and equivalent articles by those of ordinary skill in the art. Rather it is hereby intended the scope be defined by the claims appended hereto.

What is claimed is:

1. A system, comprising:
a liquid monitoring system, wherein the liquid monitoring system is externally coupled to a liquid storage container storing a liquid, wherein the liquid monitoring system comprises an electrode and a sensor, wherein the electrode generates capacitance data and the sensor generates environmental data;
a storage container monitoring computing system in communication with the liquid monitoring system over a communications network, the storage container monitoring computing system comprising computer-readable medium having computer-executable instructions stored thereon and one or more computer processors, the computer-executable instructions configured to instruct one or more computer processors to perform the following operations:
receive the capacitance data and the environmental data generated by the liquid monitoring system; and
based on the received capacitance data and the environmental data, determine an alcohol by volume (ABV) for the liquid stored in the liquid storage container.

2. The system of claim 1, wherein the liquid monitoring system is not in contact with the liquid.

3. The system of claim 1, wherein the electrode is one of a plurality of electrodes coupled to the liquid monitoring system.

4. The system of claim 3, wherein the plurality of electrodes comprises a sensing electrode and a static electrode.

5. The system of claim 4, wherein the sensor is a temperature sensor.

6. The system of claim 4, wherein the sensor is a humidity sensor.

7. The system of claim 4, wherein the sensor is a one of a plurality of sensors, wherein the plurality of sensors comprises a temperature sensor and a humidity sensor.

8. The system of claim 1, wherein the determination of the alcohol by volume for the liquid stored in the liquid storage container is based on a model according to a type of liquid being stored.

9. The system of claim 1, wherein the liquid storage container is a barrel and the liquid is distilled spirits.

10. The system of claim 9, comprising a plurality of liquid monitoring systems, wherein each of the plurality of liquid monitoring systems is externally coupled to a respective barrel of distilled spirits positioned in a rickhouse.

11. The system of claim 10, wherein each of the respective barrel of distilled spirits is a wooden storage container having a wooden end wall and each liquid monitoring system of the plurality of liquid monitoring system is coupled to the respective wooden end wall.

12. A system, comprising:
a plurality of liquid monitoring systems, wherein each of the plurality of liquid monitoring systems are coupled to a wooden end wall of a respective liquid storage container that is storing alcoholic liquids in a storage facility, wherein the liquid monitoring system comprises an electrode and a sensor, wherein the electrode generates capacitance data and the sensor generates environmental data;
a storage container monitoring computing system in communication with each of the plurality of liquid monitoring systems over a communications network, the storage container monitoring computing system comprising computer-readable medium having computer-executable instructions stored thereon and one or more computer processors, the computer-executable instructions configured to instruct one or more computer processors to perform the following operations:
receive the capacitance data and the environmental data generated by each of the plurality of liquid monitoring systems; and
for the alcoholic liquids stored in each of the plurality of liquid storage containers, determine an alcohol by volume (ABV) based on the received capacitance data and the environmental data for each respective liquid storage container.

13. The system of claim 12, wherein the liquid monitoring systems are not in contact with the alcoholic liquids.

14. The system of claim 12, wherein the electrode is one of a plurality of electrodes coupled to the liquid monitoring system, and wherein the plurality of electrodes comprises a sensing electrode and a static electrode.

15. The system of claim 12, wherein the sensor is any of a temperature sensor and a humidity sensor.

16. The system of claim 12, wherein the determination of the alcohol by volume is based on a model associated with a type of alcoholic liquid stored in the liquid storage container.

17. The system of claim 12, wherein each of the plurality of liquid storage containers is a wooden storage container having a wooden end wall and each liquid monitoring system of the plurality of liquid monitoring system is coupled to the respective wooden end wall, wherein the liquid monitoring system does not contact the alcoholic liquid.

18. A storage container monitoring computing system comprising computer-readable medium having computer-executable instructions stored thereon and one or more computer processors, the computer-executable instructions configured to instruct one or more computer processors to perform the following operations:
receiving a capacitance measurement from each of the plurality of liquid monitoring systems, wherein each of the plurality of liquid monitoring systems is coupled to a respective one of a plurality of liquid storage containers positioned within a storage facility, wherein each of the plurality of respective liquid monitoring systems comprises at least one electrode configured to measure capacitance, and each of the plurality of liquid storage containers is storing a liquid;
receiving environmental data from the storage facility, wherein the environmental data comprises temperature data;
for the liquid stored in each of the plurality of liquid storage containers, determine an alcohol by volume (ABV) based on the received capacitance data for each respective liquid storage container and the received environmental.

19. The storage container monitoring computing system of claim 18, wherein the at least one electrode comprises a sensing electrode and a static electrode.

20. The storage container monitoring computing system of claim 18, wherein the temperature data is collected by each of the plurality of respective liquid monitoring systems
